(19) United States
(12) Patent Application Publication
(10) Pub. No.: US 2003/0180282 A1
(43) Pub. Date: Sep. 25, 2003

(54) Method of treatment of thrombotic events

(76) Inventors: Victor Serebruany, Ellicot City, MD (US); Alex Malinin, Cockeysville, MD (US); Roswith Margreth Eisert, Hannover (DE)

Correspondence Address: LAHIVE & COCKFIELD, 28 STATE STREET, BOSTON, MA, 02109, US

(21) Appl. No.: 10/106,958

(22) Filed: Mar. 25, 2002

(51) Int. Cl. A61K 38/48; A61K 38/43

(52) U.S. Cl. 424/94.64; 424/94.1

(57) ABSTRACT

The present invention provides a method of treating a thrombotic or thromboembolic event in a patient by administering first a therapeutically effective amount of a thrombolytic agent and later a therapeutically effective amount of a platelet inhibitor, wherein the platelet inhibitor is administered after thrombolysis has occurred.

### BACKGROUND AND SIGNIFICANCE

It is currently believed that thrombosis plays a major role in the pathogenesis of unstable angina, acute myocardial infarction (MI) and ischemic stroke. Thrombosis begins with the rupture of the atherosclerotic plaque that exposes thrombogenic lipids and other subendothelial components, resulting in platelet adhesion, activation and aggregation, thrombin generation, fibrin deposition and the eventual formation of an occlusive clot.

Patients diagnosed with acute MI associated with ST-segment elevation, as assessed by an electrocardiogram (ECG), usually have a complete coronary occlusion when given an angiography. This is also known as a “Q-wave myocardial infarction.” Initial therapy for these patients includes prompt recanalization for the affected coronary vessel by fibrinolytic therapy or primary angioplasty to arrest the wavefront of the myocardial necrosis, preserve left-ventricular function and diminish mortality.

The benefit of fibrinolytic therapy for the treatment of acute myocardial infarction has been demonstrated in five major placebo-controlled, randomized trials that used as primary end points the reduction in short-term (3 to 5 weeks) mortality (Lancet 1(8478):397-402, (1986); Lancet 2(8607):349-360, (1988); N. Engl. J. Med. 14(23):1465-1471, (1986); Lancet 1(8585):545-549, (1988); Wilcox, R G et al. (1988) Lancet 2(8610):525-530). These trials showed an overall reduction in mortality of 27%. A further benefit was achieved with the addition of the “accelerated” alteplase (t-PA) regimen in the Global Utilization of Streptokinase and Tissue plasminogen activator (GUSTO) trial (N. Engl. J. Med. 329(10):673-682, (1993)). However, despite the improvements demonstrated in these trials, current fibrinolytic regimens have several drawbacks, including the failure to induce early and sustained reperfusion in 40% to 50% of patients, reocclusion in 10% to 20% of patients, and intracranial hemorrhage in 1% to 3% of patients (Topol, E J et al. (2000) Circulation 102:1761-1765; Eikelboom J W et al. (2001) JAMA 285:444-450; Wienbergen, H. et al. (2001) Am. J. Cardiol. 87:782-785, A8).

Other major advances in the acute therapy for myocardial infarction include the coadministration of aspirin with thrombolytic therapy. A meta-analysis of 32 studies of aspirin has shown a significant effect on reocclusion and recurrent ischemia, with an approximate halving of frequency of these adverse end points (Roux, S. et al. (1992) J. Am. Coll. Cardiol. 19(3):671-677). As a result, the American College of Cardiology (ACC) and the American Heart Association (AHA) now recommend aspirin as a “first line” medication that should be administered to all patients with acute coronary syndrome with the exception of a few contraindications (J. Am. Coll. Cardiol. 28:1328-1428, (1996)). As an alternative to aspirin, ADP receptor blockers such as ticlopidine and clopidogrel can be used as an alternative or addition to aspirin in these situations.

The potential role of glycoprotein IIb/IIIa receptor inhibitors for use in acute MI therapy has also been investigated. Pharmacological compounds directed against glycoprotein IIb/IIIa block the receptor, and thus prevent the binding of circulating adhesion molecules, and fibrinogen, thus potently inhibiting platelet aggregation. Intravenous agents of this class include the chimeric Fab monoclonal antibody fragment abciximab (Reo-Pro®, Johnson&Johnson), the peptide inhibitor eptifibatide (Integrilin®, COR Therapeutics), the nonpeptide mimetic tirofiban (Aggrastat®, Merck & Co.) and lamifiban (Hoffman-La Roche, Inc.). Aciximab, eptifibatide and tirofoban (http://www.fda.gov/cber/label/abcicen021898LB.pdf; http://www.fda.gov/cder/foi/label/1998/207181bl.pdf; http://www.fda.gov/cder/foi/label/1998/209121bl.pdf) are indicated by the Food and Drug Administration (FDA) for the treatment of acute coronary syndrome, including patients who are to be managed with medication or percutaneous coronary intervention (PCI).

Based on the success in using aspirin and glycoprotein IIb/IIIa receptor inhibitors the combination therapeutic strategy for fibrinolytic agents and platelet inhibitors is currently recommended, and being utilized. This involves concomitant use of thrombolytics and glycoprotein IIb/IIIa inhibitors as adjuvant therapy to full-dose fibrinolytics and the second involves thrombolytics and glycoprotein IIb/IIIa receptor inhibitors as adjunctive therapy with reduced-dose fibrinolytic agents. The Thrombolysis in Myocardial Infarction (TAMI-8), Integrilin to Minimize Platelet Aggregation and Coronary Thrombosis in Acute Myocardial Infarction (IMPACT-AMI) and Platelet Aggregation Receptor Antagonist Dose Investigation Reperfusion Gain in Myocardial Infarction (PARADIGM) trials tested the concomitant use of thrombolytics and glycoprotein IIb/IIIa inhibitors as adjuvant therapy to full-dose fibrinolytics (Kleinman, N S et al. (1991) Circulation 84:II-522 (Abstract); Ohman E M et al. (1997) Circulation 95(4):846-854; J. Am. Coll. Cardiol. 32(7):2003-2010, (1998)). Despite the improved potency of the coronary artery, these strategies were associated with a significant risk of bleeding (7.1% of major hemorrhage in IMPACT-AMI study). Moreover, the incidence of death and reinfarction was 8.0% for all patients randomized to eptifibatide (IMPACT-AMI) versus 7.3% in the placebo group. The trial employing the combination of eptifibatide and full-dose streptokinase was terminated early because of marked excess risk of hemorrhage (Ronner, E. et al. (1998) J. Am. Coll. Cardiol. 31:191A). These discouraging data evolved another strategy, namely to reduce the dose of both agent classes in order to diminish incidence of bleeding complications. Contrarily, the outcomes of the Thrombolysis in Myocardial Infarction (TIMI-14) (abciximab with streptokinase or reduced dose of alteplase) and the Strategies for Patency Enhancement in the Emergency Department (SPEED) (abciximab with low-dose reteplase) trials resulted in a significantly increased rate of bleeding. Specifically, hemorrhaging occurred in 7% of patients treated with abciximab plus alteplase, 10% of patients treated with streptokinase plus abciximab and 9.8% of patients treated with abciximab plus reduced dose of reteplase. On the other hand, only 3.7% of patients treated with reteplase alone suffered hemorrhaging. In the most recent and largest GUSTO-V trial (16,588 patients), the combination of half-dose reteplase with full-dose abciximab failed to show any advantages over using reteplase alone (rate of death 5.6% versus 5.9%, respectively) (The GUSTO V Investigators, (2001) Lancet 357:1905-1914). The increased risk of hemorrhagic bleedings was also observed among patients over the age of 75 years who were treated with combination of half-dose reteplase with full-dose abciximab (1.2% with reteplase alone versus 2.1% with combination therapy).

Therefore, additional insight into treatments which promote thrombolysis and help reduce platelet aggregation while minimizing or eliminating the risk of hemorrhage will be of great benefit in treating patients suffering from vascular thrombotic events such as acute myocardial infarction and ischemic stroke.

### SUMMARY OF THE INVENTION

The invention relates, at least in part, to the treatment of a thrombotic or thromboembolic event in a patient using a thrombolytic agent and a platelet inhibitor(s).

One aspect of the invention features a method of treating a thrombotic or thromboembolic event in a patient in need of such treatment comprising administering a therapeutically effective amount of a thrombolytic agent and administering a therapeutically effective amount of a platelet inhibitor, wherein the antiplatelet agent(s) is administered to the patient not concomitantly with said thrombolytic agent, but after thrombolysis has occurred.

Another aspect of the invention features the concomitant administration of an anticoagulant compound with the thrombolytic agent, the platelet inhibitor, or both. Preferably, the anticoagulant compound is selected from the group consisting of unfractionated heparin, heparin and hirudin.

In one embodiment of the invention, the thrombotic or thromboembolic event is selected from the group consisting of unstable angina, acute myocardial infarction, ischemic stroke, acute coronary ischemic syndrome, thrombosis, thromboembolism, deep vein thrombosis, arterial thrombosis of any vessel, catheter thrombotic occlusion, thrombotic occlusion and reocclusion, transient ischemic attack, first or subsequent thrombotic stroke.

In another embodiment of the invention, the thrombolytic agent is selected from the group consisting of streptokinase, alteplase, reteplase, monteplase, lanoteplase, saruplase, pro-urokinase, urokinase, pro-urokinase, staphylokinase, tenecteplase, and anisoylated plasminogen-streptokinase activator complex.

In another embodiment of the invention, the platelet inhibitor is selected from the group consisting of aciximab, eptifibatide, tirofoban, lamifiban, aspirin, ticlopidine, clopidogrel, dipyridamole, aggrenox®, or selective serotonin reuptake inhibitor.

In another embodiment of the invention, one or more platelet inhibitors are preferably administered between about 6 to 24 hours, more preferably between about 12 to 24 hours, even more preferably between 18 to 24 hours, and most preferably between about 20 to 24 hours after thrombolysis has occurred.

Another aspect of the invention features administering the thrombolytic agent multiple times.

### DETAILED DESCRIPTION OF THE INVENTION

The instant invention relates to a novel dual therapy for the treatment of a thrombotic or thromboembolic event in a patient comprising the administration first of a therapeutically effective amount of a thrombolytic agent with the delayed administration of a therapeutically effective amount of a platelet inhibitor(s), wherein the platelet inhibitor(s) is administered to the patient after thrombolysis has occurred.

The term, “thrombotic or thromboembolic event,” means any disorder that involves a blockage or partial blockage of an artery or vein with a thrombosis. A thrombotic or thromboembolic event occurs when a clot forms and lodges within a blood vessel. The clot may fully or partially block the blood vessel causing a thrombotic disorder such as a heart attack or stroke. Examples of thrombotic or thromboembolic events, as used herein include, but are not limited to, thrombotic disorders such as acute myocardial infarction, unstable angina, ischemic stroke, acute coronary syndrome, pulmonary embolism, transient ischemic attack, thrombosis (e.g. deep vein thrombosis, thrombotic occlusion and re-occlusion and peripheral vascular thrombosis) and thromboembolism. A thrombotic or thromboembolic event also includes first or subsequent thrombotic stroke, acute myocardial infarction, which occurs subsequent to a coronary intervention procedure, or thrombolytic therapy.

As used herein, the term “thrombolytic agent” refers an agent that is capable of inducing reperfusion by dissolving, dislodging or otherwise breaking up a clot. Reperfusion occurs when the clot is dissolved and blood flow is restored. Some widely used thrombolytic agents include recombinant tissue plasminogen activator, such as alteplase (also known as t-PA or Activase®, Genentech, Inc.), other forms of tissue plasminogen activator, such as reteplase (also known as r-PA or retavase®, Centocor, Inc.) and tenecteplase (also known as TNK™, Genentech, Inc.), streptokinase (also known as Streptase®, AstraZeneca, LP), pro-urokinase (Abbott Laboratories), urokinase (Abbott Laboratories), lanoteplase (Bristol-Myers Squibb Company), monteplase (Eisai Company, Ltd.), saruplase (also known as r-scu-PA and rescupase™, Grunenthal GmbH, Corp.), staphylokinase, and anisoylated plasminogen-streptokinase activator complex (also known as APSAC, Anistreplase and Eminase®, SmithKline Beecham Corp.). Thrombolytic agents also include other genetically engineered plasminogen activators.

As used herein, the term “thrombolysis” means the resolution or prevention of platelet activation within the vascular bed and the dissolution of blood clots through fibrinolysis. It is therefore a more encompassing term than is fibrinolysis. Assays to measure thrombolysis are well known in the art and are discussed in detail later.

The term “acute myocardial infarction” includes myocardial infarction, which results from occlusion of a coronary artery. As used herein, the term “acute myocardial infarction” is intended to include both Q-wave and non-Q-wave myocardial infarction.

“Acute coronary ischemia” refers to local ischemia due to mechanical obstruction, e.g. arterial narrowing, of the blood supply to the heart. The obstruction may be caused by spasm of the artery or by atherosclerosis with acute clot formation. The obstruction results in damaged tissue and a permanent loss of contraction of this portion of the heart muscle. The condition is also referred to as myocardial ischemia and is characterized by inadequate circulation of blood to the myocardium, usually as a result of coronary artery disease. Ischemia of the heart muscle is evidenced by a pain in the chest often radiating from the precordium to the left shoulder and down the arm (angina pectoris) and is caused by coronary disease.

As used here in, the term “patient in need of such treatment” includes a patient of any age who present within 6 hours of symptom onset with more than 30 minutes of continuous symptoms of acute myocardial infarction and who, by electrocardiogram, have demonstrated at least 1 mm of ST-segment elevation in 2 or more limb leads or at least 2 mm ST segment elevation in 2 or more contiguous precordial leads or bundle branch block.

The term “patient” includes mammals, especially humans, who take a platelet inhibitor in combination with a thrombolytic agent for any of the uses described herein.

As used herein, the term “platelet inhibitor” is intended to include all pharmaceutically acceptable salt, ester and solvate forms, including hydrates, of compounds which have platelet inhibitory activity as well as pro-drug forms. Such pro-drugs are compounds that do not have platelet inhibitory activity outside the body but become active as inhibitors after they are administered to the patient. Therefore the use of such salts, esters solvate forms and pro-drugs of antiplatelet agents is included within the scope of this invention.

The term “pharmaceutically acceptable salts” refers to non-toxic salts of the compounds employed in this invention which are generally prepared by reacting the free acid with a suitable organic or inorganic base. Examples of salt forms of platelet aggregation inhibitors may include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylbromide, methylnitrate, methylsulfate, mucate, napsylate, nitrate, oleate, oxalate, pamaote, palmitate, panthothenate, phosphate/diphosphate, polygalacturonate, potassium, salicylate, sodium, stearate, subacetate, succinate, tannate, tartrate, teoclate, tosylate, triethiodide, and valerate.

Other platelet inhibitors suitable for use in the present invention include clopidogrel (Plavix®, Sanofi—Bristol Myers Squibb) and ticlopidine (Ticlid®, Roche Laboratories), both of which block ADP-induced platelet aggregation, as well as dipyridamole (Persantine®, Boehringer Ingelheim), a platelet adhesion inhibitor, the glycoprotein IIb/IIIa antagonists aciximab (ReoPro®, Eli Lilly & Co.), eptifibatide (Integrilin®, Cor Therapeutics), tirofoban (Aggrastat®, Merck & Co., Inc.) and lamifiban, as well as aspirin or aspirin-dipyridamole combination (Aggrenox®, Boehringer Ingelheim) and selective seretonin reuptake inhibitors.

As used herein, the term “anticoagulant compound” refers to any compound that decreases the clotting ability of the blood and therefore helps to prevent harmful clots from forming in the blood vessels. These compounds are also commonly referred to as blood thinners. Although these compounds usually do not dissolve clots that already have formed, they may prevent the clots from becoming larger and causing more serious problems. Anticoagulant compounds suitable for use in this invention include, but are not limited to, unfractionated heparin, heparin and hirudin.

Therapeutic Methods

This combination therapy includes the administration of a thrombolytic agent in its own separate pharmaceutical dosage formulation followed by the administration of a platelet inhibitor in its own separate pharmaceutical dosage formulation after thrombolysis has occurred. The thrombolytic agent can be administered intravenously or parenterally. The platelet inhibitor can be administered orally, intravenously, transdermally (for example using an iontophoretic patch), intraocularly, intranasally or by other routes known to those skilled in the medical arts, taking into account that certain platelet inhibitors are developed for oral administration while others may be developed for non-oral routes such as intravenous administration. Ticlopidine, clopidogrel, aspirin and dipyridamole are administered orally.

In one aspect of the invention, separate dosage formulations are used. For example, the thrombolytic agent and the platelet inhibitor are administered at separately staggered times, i.e. the platelet inhibitor is administered after thrombolysis has occurred. Preferably, the platelet inhibitor is administered between about 6 hours and 24 hours after thrombolysis has occurred, more preferably between about 12 hours and 24 hours after thrombolysis has occurred and most preferably between about 20 hours and 24 hours after thrombolysis has occurred.

In another aspect of the invention, the thrombolytic agent is administered multiple times in order to achieve thrombolysis. The number of doses administered will depend on the type and severity of the thrombotic or thromboembolic condition to be treated. This determination can be made by one skilled in the art and is within the scope of the invention.

Therapeutically effective amounts of the platelet inhibitors and the thrombolysis agents are suitable for use in the compositions and methods of the present invention. The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The dosage regimen utilizing a thrombolytic agent in combination with a platelet inhibitor is selected in accordance with a variety of factors including type, species, age, weight, sex and medical condition of the patient; the severity of the condition to be treated; the route of administration; the renal and hepatic function of the patient; and the particular compound or salt or ester thereof employed. Since two different active agents are being used together in a combination therapy, the potency of each of the agents and the enhanced effects achieved by combining them together must also be taken into account. A consideration of these factors is well within the purview of the ordinarily skilled clinician for the purpose of determining the therapeutically effective amounts of the drug combination needed to prevent, counter, or arrest the progress of the condition.

Dosage information for thrombolytic agents is well known in the art, since several thrombolytic agents are marketed in the U.S. The daily dosage amounts for alteplase, reteplase and tenecteplase will vary depending on the weight of the patient, the type of thrombotic event that has occurred, as well as other factors noted above. Moreover, dosages may be given in a single dose or as separate doses given at different times and the amount of thrombolytic agent may remain constant or vary with each dose. These are determinations that can be made by one with skill in the art and are within the scope of the invention. Typically, intravenous doses of alteplase will range between about 15 mg to 100 mg, preferably between about 20 mg to 80 mg for a patient weighing over 65 kg. Intravenous doses of alteplase will range between about 0.1 mg/kg to 2.0 mg/kg, preferably between about 0.4 mg/kg to 1.3 mg/kg for patients weighing less than 65 kg. Intravenous doses of reteplase will range between about 1 mg and 25 mg, preferably between about 5 mg and 10 mg. Intravenous doses of tenecteplase will range between about 50 mg/day to 400 mg/day, preferably between about 150 mg/day and 325 mg/day.

Oral dosages of glycoprotein IIb/IIIa receptor antagonists when used for the indicated effects, will typically range between about 0.001 mg per kg of body weight per day (mg/kg/day) to about 50 mg/kg/day and preferably 0.005-20 mg/kg/day and most preferably 0.005-10 mg/kg/day. Suitable oral tablets and capsules contain between 0.1 mg and 5 g, preferably between 0.5 mg and 2 g, most preferably between 0.5 mg and 1 g, for example, 0.5 mg, 1 mg, 5 mg, 10 mg, 50 mg, 150 mg, 250 mg, or 500 mg of glycoprotein IIb/IIIa receptor antagonist. Oral administration may be in one or divided doses of two, three, or four times daily. A single daily dose is preferred.

Intravenously, the most preferred doses for platelet inhibitors will range from about 0.5 pg to about 5 mg/kg/minute during a constant rate infusion, to achieve a plasma level concentration during the period of time of administration of between 0.1 ng/ml and 1 ug/ml.

Dosage amounts for ticlopidine are described in the Physicians' Desk Reference.

Dosage amounts of aspirin for the indicated effects are known to those skilled in medical arts, and generally range from about 75 mg to about 325 mg per day. For example, a formulation may contain 75 mg, 81 mg, 160 mg, 250 mg, or 325 mg of aspirin.

Suitable oral formulations of clopidogrel may contain from 25 mg to 500 mg, preferably from 75 mg to 375 mg, and most preferably from 75 mg to 150 mg of clopidogrel. For example, the formulation may contain 25 mg, 50 mg, 75 mg, 150 mg, 250 mg, or 500 mg of clopidogrel. Oral administration may be in one or divided doses of two, three, or four times daily. A single daily dose is preferred. Dosage amounts for ticlopidine and for dipyridamole are described in the Physicians' Desk Reference. Dosage amounts of aspirin for the indicated effects are known to those skilled in medical arts, and generally range from about 75 mg to about 325 mg per day. For example, a formulation may contain 75 mg, 80 mg, 160 mg, 250 mg, or 325 mg of aspirin.

Suitable intravenous compositions for the platelet inhibitors include bolus or extended infusion. Such oral and intravenous compositions are known to those of ordinary skill in the pharmaceutical arts (see, e.g., Remington's Pharmaceutical Sciences, Mack Publishing Co., Easton, Pa).

Suitable Assays for Thrombolysis and Platelet Aggregation

A number of assays are known in the art for measuring thrombolysis and components of the thrombolytic system. For example, U.S. Pat. No. 5,612,187 provides a clot time determining device and method for determining the time necessary for a test fluid to lyse a clot. This test can provide a measure of the action of plasminogen activators and plasmin in the blood. The patent further contains a discussion on the complex interplay between and balance of the coagulation system, which when working properly forms clots to protect the body from loss of blood, and the fibrinolytic system, which when working properly removes clots when they are no longer needed. The patent further refers to commercially available kits for immunologic detection of fibrin degradation products, permitting a measure of the function of the fibrinolytic system. U.S. Pat. No. 5,587,159 teaches assays for fibrinolytic activity and plasminogen activation, as well as direct and indirect assays for plasmin formation.

Use of thrombolytics affects the clotting time of blood in a patient during and for some time after the administration of the agent. It is currently recommended that where time permits, the use of such agents be preceded by obtaining a hematocrit, platelet count, a thrombin time (TT), activated partial thromboplastin time (APTT), or prothrombin time. Coagulation tests and measures of fibrinolytic activity can be made during the administration of the agents if desired. All of these tests are known in the art.

Coagulation assay procedures are described in, for example, Smith, et al., (Smith et al. (1988) Thrombosis Research, 50:163-174). U.S. Pat. Nos. 5,688,813 and 5,668,289 teach assaying coagulation time and related determinations in murine and canine models of arterial injury and of coronary artery thrombosis. U.S. Pat. Nos. 4,861,712; 4,910,510; 5,059,525; and 5,580,744 describe test articles suitable for monitoring blood coagulation. U.S. Pat. No. 4,756,884 describes a capillary flow device for measuring blood characteristics, including prothrombin time. A platelet aggregation assay, a platelet-fibrinogen binding assay, and a thrombolytic assay, are all taught in U.S. Pat. No. 5,661,159. Simple tests, such as rocking a blood sample in a test tube and timing the period until the blood clots, in the presence or absence of known or potential anti-coagulants, as well as whole blood aggregation techniques and flow cytometric analysis of appropriate adhesion surface markers are also known. Whichever assay is employed, the assays should be performed serially and as often as possible in order to assure accurate measurements.

Several tests are also known to one skilled in the art to evaluate the activity of a platelet aggregation inhibitor. For example, one test which is commonly used to evaluate IIb/IIIa receptor antagonist activity is based on evaluation of inhibition of ADP-stimulated platelets. Aggregation requires that fibrinogen bind to and occupy the platelet fibrinogen receptor site. Inhibitors of fibrinogen binding inhibit aggregation. In the ADP-stimulated platelet aggregation assay, human platelets are isolated from fresh blood, collected into acid citrate/dextrose by differential centrifugation followed by gel filtration on Sepharose 2B in divalent ion-free Tyrode's buffer (pH 7.4) containing 2% bovine serum albumin.

Platelet aggregation is measured at 37° C. in a Chronolog aggregometer. The reaction mixture contains gel-filtered human platelets (26×108 per ml), fibrinogen (100 micrograms per ml (μg/ml)), Ca²⁺ (1 mM), and the compound to be tested. The aggregation is initiated by adding 10 mM ADP 1 minute after the other components are added. The reaction is then allowed to proceed for at least 2 minutes. The extent of inhibition of aggregation is expressed as the percentage of the rate of aggregation observed in the absence of inhibitor. The IC₅₀ is the dose of a particular compound inhibiting aggregation by 50% relative to a control lacking the compound.

Success of thrombolysis can be determined by using a number of techniques that are well known in the art. For example, thrombolysis can be evaluated by angiography, scintigraphy, electrocardiogram (ECG), patient condition (i.e. assessment of symptom relief) and, indirectly, by measuring the plasma levels of myocardial necrosis biomarkers.

Other features, advantages and embodiments of the invention will be apparent from the following examples which are meant to be illustrative, and therefore, not limiting in any way.

### Examples

### A. Example 1

### Heterogeneity of Platelet Aggregation and Major Surface Receptor Expression in Patients Presenting with Acute Myocardial Infarction.

This example compares the platelets of acute myocardial infarction patients before thrombolysis and the administration of adjunctive therapy, who were later enrolled in the GUSTO-III trial with a group of healthy patients (controls).

Materials and Methods

Twenty three patients admitted to the emergency room of St. Agnes Hospital, or to the Union Memorial Hospital between July and December of 1996 with a diagnosis of acute myocardial infarction who were subsequently enrolled in the GUSTO-III trial were studied. The inclusion and exclusion criteria of GUSTO-III have been previously reported. In summary, patients of any age who presented within 6 hours of symptom onset with more than 30 minutes of continuous symptoms of AMI, and who by 12-lead electrocardiogram demonstrated at least 1 mm of ST segment elevation in 2 or more limb leads or at least 2 mm ST segment elevation in 2 or more contiguous precordial leads or left bundle branch block were included in this trial. Patients were excluded if they had a history of bleeding diathesis, history of stroke, major surgery or significant trauma in the past six weeks, and hypertension of more than 200/110 mm Hg. Blood samples for PA, and for flow cytometric studies were taken in the emergency room before the administration of reperfusion therapy.

For a control population, ten subjects (age 21-43; 6 males, 4 females) without a history of bleeding disorders, cardiovascular disease, and for at least two weeks were free of pharmacologic agent use, were enrolled in the study. None of the controls smoked, or exhibited hypertension, diabetes, or an abnormal hematocrit. The lipid status of the controls was uncertain. All subjects underwent blood sampling after at least 30 minutes of rest and 2 or more hours of fasting. Blood was drawn between 8 and 10 a.m. in order to avoid any diurnal influence and sampled from an antecubital vein, as in the experimental group. To avoid possible observer bias, blood samples were coded and blinded. Sampling procedures, platelet aggregation (PA), and flow cytometric studies were performed by individuals unaware of the protocol.

Aggregation studies were performed as previously described Serenbruany, V. L. et al. (Serenbruany, V L (1996) J. Cardiovasc. Pharm. 28:175-181). In brief, citrate solution and whole blood were immediately mixed 1:9 and then centrifuged at 1200 g for 2.5 minutes in order to obtain platelet-rich plasma (PRP) which was kept at room temperature for use within 1 hour. Platelet counts were determined for each PRP sample with a Coulter Counter ZM (Coulter Co., Hialeah, Fla.). Platelet numbers were adjusted to 3.50×10⁸/ml with homologous platelet-poor plasma. PA was induced by 5 μM ADP; 10 μM ADP; 1 μg/ml collagen; 2 U/ml thrombin, and 1.25 mg/ml ristocetin. All agonists were obtained from Chronolog Corporation (Hawertown, Pa.). PA studies were performed using a Chrono-Log Whole Blood Lumi-Aggregometer (model 560-Ca). PA was expressed as a percentage of light transmittance change from the baseline using platelet-poor plasma as a reference at the end of the recording time. PA curves were recorded for 4 minutes and analyzed according to internationally established standards (see, e.g. Ruggeri, Z M (1994) Semin. Hemat. 31:229-239).

For flow cytometric analysis, venous blood (8 ml) was collected in a plastic tube containing 2 ml of acid-citrate-dextrose (ACD) (7.3 g citric acid, 22.0 g sodium citrate ×2H₂O and 24.5 glucose in 1000 ml distilled water) and mixed well. The blood-ACD mixture was centrifuged at 1000 r.p.m. for 10 minutes at room temperature. The upper ⅔ of the platelet-rich plasma (PRP) was then collected and adjusted to pH=6.5 by adding ACD. The PRP was then centrifuged at 3000 r.p.m. for 10 minutes. The supernatant was removed and the platelet pellet was gently resuspended in 4 cc of the washing buffer (10 mM Tris/HCl, 0.15 M NaCl, 20 mM EDTA, pH=7.4). Platelets were washed 4 times in the washing buffer, and an additional 4 times in TBS (10 mM Tris, 0.15 M NaCl, pH=7.4). All cells were then divided into ten plastic tubes. Nine portions of washed platelets were incubated with 5 μl fluorescein isothiocyanate (FITC)-conjugated antibodies in the dark at +4° C. for 30 minutes, and one part remained unstained and served as a negative control. Surface antigen expression was measured with monoclonal murine anti-human antibodies: CD9 (p24); CD41a (IIb/IIIa); CD42B (Ib); CD61(IIIa) (DAKO Corporation, Carpinteria Calif., U.S.A.); CD49b (VLA-2, or Ia-IIa); CD62p (P selectin); CD31 (PECAM-1); CD 41b (IIb); and CD51/CD61 (vitronectin receptor) (PharMingen, San Diego, Calif., U.S.A.). After incubation, the cells were washed three times with TBS and resuspended in 0.25 ml of 1% paraformaldehyde. Samples were analyzed on a flow cytometery with laser output of 15 mw, excitation at 488 nm, and emission detection at 530±30 nm (Becton Dickinson FACScan, San Jose, Calif.). The instrument was calibrated daily with fluorescence beads (CaliBRITE; Becton Dickinson) and measured FITC-conjugated fluorescence intensity. All parameters were obtained using four decade logarithmic amplification. The data was collected and stored in list mode, and then analyzed using CELLQuest™ (version 1.2.2) software.

The schedules for blood drawing, sample preparation, and processing were critical issues of the study design and were monitored by an independent observer. The actual timing of blood collection for the baseline sample was 9.5±1.4 minutes before the start of thrombolytic therapy. Samples were processed and stained within 1 hour after blood drawing. Platelet aggregometry was performed within 2 hours of the blood draw. Stained samples fixed with paraformaldehyde were stored in the refrigerator at +4° C. for no more than 48 hours before flow cytometic analysis.

For statistical analysis, a post hoc comparison using the Bonferroni t-test was performed to identify specific differences in PA, and receptor expression between AMI patients and controls. A Mann-Whitney U test was used to analyze nonparametric data. Data are expressed as mean±SD; (range); and p<0.05 was considered significant. Differences between individual flow cytometric histograms were assessed using the Smirnov-Kolmogorov test incorporated in the CELLQuest software.

Results

Platelet aggregation (PA) in response to each agonist was determined in every patient and control. PA was significantly higher in AMI patients when induced with both concentrations of ADP, by thrombin, and by ristocetin. There were no statistically significant differences between the groups to the extent of PA when collagen was used as an agonist.

Although most of the patients exhibited increased PA, individual data reveal a consistent heterogeneity of platelet function. PA induced by ADP 5 μM was within the normal range in nine AMI patients. When 10 μM ADP was used to induce PA, normal range results were also observed in nine patients. None of the patients were above the normal range when PA was induced by collagen. Thrombin—stimulated PA was within the control levels in eleven AMI patients. Ristocetin was the most significant discriminating stimulus between the two groups, with the least heterogeneous response, however, four patients did not differ from controls.

Next, flow cytometry of fluorescence expression of single-labeled major platelet surface receptors in AMI patients and controls were analyzed. Although there was an overall slight increase in platelet antigen expression in AMI patients, the data revealed no statistically significant differences for five major receptors between AMI patients and controls. A significant elevation of ADP-, thrombin-, and ristocetin-induced PA, and the surface expression of P-selectin (CD 62p), and PECAM-1 (CD35) occurred in AMI patients prior to reperfusion therapy, as compared with healthy controls. Although glycoprotein IIb/IIIa receptor expression was slightly increased, it did not differ significantly from controls. No statistically significant differences were observed for collagen-induced PA, and surface expression of platelet 24 (CD9); glycoprotein IIIa (CD 61); glycoprotein IIb/IIIa (CD41b/CD61); VLA-2 (CD 49b); and platelet vitronectin receptor (CD51/CD 61). Moreover, we observed a significant inhibition in glycoproteins IIb (CD 41b) and Ib (CD 42b) expression in AMI patients before thrombolysis as compared with controls. The data of the individual platelet antigen expression reveals that at least one third of AMI patient population were within the normal, or below normal range.

### B. Example 2

### Effects of Reteplase and Alteplase on Platelet and Major Receptor Expression During the First 24 Hours of Acute Myocardial Treatment

This example demonstrates the immediate and early platelet-related effects of alteplase (t-PA) and reteplase (r-PA) in acute myocardial infarction patients enrolled in the GUSTO-III trial. Platelet aggregation (PA) was measured in response to multiple agonists and the major surface receptor expression was determined by flow cytometry at prespecified time points following attempted reperfusion.

Materials and Methods

Twenty-three consecutive patients admitted to the emergency rooms of St. Agnes Hospital, or the Union Memorial Hospital between July and December of 1996 with a diagnosis of acute myocardial infarction were included. All patients were enrolled in the randomized trial of Reteplase (r-PA) versus accelerated Alteplase (t-PA) for the treatment of acute myocardial infarction, (GUSTO-III trial). Patients of any age who presented within 6 hours of symptom onset with more than 30 minutes of continuous symptoms of AMI, and who by 12-lead electrocardiogram had demonstrated at least 1 mm of ST segment elevation in 2 or more limb leads or at least 2 mm ST segment elevation in 2 or more contiguous precordial leads or bundle branch block were included in this trial. Patients were excluded if they had a history of bleeding diathesis, stroke, major surgery or significant trauma in the past six weeks, and hypertension more than 200/110 mm Hg.

Blood samples for PA, and for flow cytometric studies were taken at prespecified intervals as follows: in the emergency room immediately before administration of the thrombolytic therapy and then in the coronary care unit at 3 hours, 6 hours, 12 hours, and finally at 24 hours after initiation of the r-PA or t-PA therapy.

Thirteen patients randomized to r-PA received a double bolus, 10+10 MU thirty minutes apart. Ten patients received t-PA in the accelerated regimen as a 15 mg bolus, then 0.75 mg/kg over 30 minutes, and then 0.50 mg/kg over 60 minutes. During the baseline sampling every patient had received 325 mg of aspirin and each day thereafter, at least 5,000 U of intravenous heparin. Following the administration of thrombolytic therapy all patients received a continuous infusion of heparin for the first 24 hours following thrombolysis as recommended in the GUSTO-III protocol.

The schedule of blood drawing, sample preparation and processing were critical issues of the study design, and were monitored by an independent observer. The actual timing of blood collection for the baseline sample was 9.5±1.4 minutes before the start of thrombolytic therapy; 174.6±21.8 minutes for the 3 hours sample; 371.1±24.2 minutes for 6 hours sample; 709.4±17.8 minutes for 12 hours sample; and 1402.9±18.8 minutes for 24 hours sample. Samples were processed within one hour after blood drawing. Four patients (3 r-PA, and 1 t-PA) did not complete the protocol at the various time points. The reasons for early termination were as follows: patient transfer for emergency coronary angioplasty (3 patients), and inability to obtain blood sample (1 patient). Twenty three baseline samples; twenty two samples collected at 3 hours; twenty samples collected at 6 hours; twenty samples collected at 12 hours; and nineteen samples collected at 24 hours were included in the study analysis.

Citrate and whole blood were immediately mixed 1:9 and then centrifuged at 1200 g for 2.5 minutes in order to obtain platelet-rich plasma (PRP) which was kept at room temperature for use within 1 hour. Platelet counts were determined for each PRP sample with Coulter Counter ZM (Coulter Co., Hialeah, Fla.). Platelet numbers were adjusted to 3.50×10⁸/ml with homologous platelet-poor plasma. PA was induced by 5 μM ADP; 10 μM ADP; 1 g/ml collagen; 1 mg/ml thrombin, and 1.25 mg/ml ristocetin. All agonists were obtained from Chronolog Corporation (Hawertown, Pa.). PA studies were performed using a Chronolog Whole Blood Lumi-Aggregometer (model 560-Ca). PA was expressed as the maximum percentage of light transmittance change from the baseline using platelet-poor plasma as a reference at the end of the recording time. PA curves were recorded for 4 minutes and analyzed according to internationally established standards (see, e.g. Ruggeri, Z. M. (1994) Semin. Hemat. 31:229-239).

For flow cytometric analysis, venous blood (8 ml) was collected in a plastic tube containing 2 ml of acid-citrate-dextrose (ACD) (7.3 g citric acid, 22.0 g sodium citrate ×2H₂O and 24.5 glucose in 1000 ml distilled water) and mixed well. The blood-ACD mixture was centrifuged at 1000 r.p.m. for 10 minutes at room temperature. The upper ⅔ of the platelet-rich plasma (PRP) was then collected and adjusted to pH=6.5 by adding ACD. The PRP was then centrifuged at 3000 r.p.m. for 10 minutes. The supernatant was removed and the platelet pellet was gently resuspended in 4 cc of the washing buffer (10 mM Tris/HCl, 0.15 M NaCl, 20 mM EDTA, pH=7.4). Platelets were washed 4 times in the washing buffer, and an additional four times in TBS (10 mM Tris, 0.15 M NaCl, pH=7.4). All cells were then divided into ten plastic capped tubes. Nine portions of washed platelets were incubated with 5 μl fluorescein isothiocyanate (FITC)-conjugated antibodies in the dark at +4° C. for 30 minutes, and one part remained unstained and served as a negative control. Surface antigen expression was measured with monoclonal murine anti-human antibodies: CD9 (p24); CD41a (IIb/IIIa, (IIb(3); CD42b (Ib); CD61(IIIa) (DAKO Corporation, Carpinteria Calif.); CD49b (VLA-2, or (2(1); CD62p (P selectin); CD31 (PECAM-1); CD 41b (IIb); and CD51/CD61 (vitronectin receptor, (v(3) (PharMingen, San Diego Calif.). After incubation, the cells were washed three times with TBS and resuspended in 0.25 ml of 1% paraformaldehyde. Samples were stored in the refrigerator at +4° C., and analyzed on a Becton Dickinson FACScan™ flow cytometry with laser output of 15 mw, excitation at 488 mm, and emission detection at 530±30 nm. The instrument was calibrated daily with fluorescence beads (CaliBRITE™; Becton Dickinson) and measured FITC-conjugated fluorescence intensity. All parameters were obtained using four decade logarithmic amplification. The data was collected and stored in list mode, and then analyzed using CELLQuest™ (version 1.2.2) software.

Statistical analyses was performed using a post hoc t-test with the Bonferroni correction was performed to identify specific differences in platelet aggregation, and receptor expression between AMI patients treated with r-PA and t-PA, and between different time points within each group. A Mann-Whitney U test was used to analyze non-parametric data. Normally distributed data are expressed as mean±SD; p<0.05 was considered significant, and skewed data as median (range). Differences between individual flow cytometric histograms were assessed using the Smimov-Kolmogorov test incorporated in the CELLQuest™ software.

Results

Patient groups were of similar demographics. Three more patients received aspirin on a daily basis in the r-PA group. Groups were also similar in background medications, AMI locations, and other baseline clinical and laboratory characteristics during thrombolysis. Three patients (r-PA-2; t-PA-1) had persistent chest pain and ST elevation and underwent immediate angiography which revealed absence of reperfusion. Two patients (r-PA-1; t-PA-1) developed recurrent ischemia in the first twenty-four hours and also underwent emergency angiography. There were no statistically significant differences in platelet aggregation between the baseline and any other time point in the t-PA group. However, the r-PA treated group revealed significant changes in platelet aggregation induced by all agonists except ristocetin.

After 5 μM ADP treatment, platelets aggregated significantly stronger in the r-PA group at 12 hours (p=0.04) and especially after 24 hours (p=0.007) after thrombolysis when compared with the t-PA treated group. Significant (when compared to baseline) inhibition of platelet aggregability was observed at 3 hours (p=0.02), and at 6 hours (p=0.02) after r-PA therapy.

After 10 μM ADP treatment, platelets exhibited similar patterns to those observed following 5 μM ADP-induced aggregation and were significantly more active in the r-PA group after 24 hours (p=0.02) as compared with the t-PA treated group. Significant inhibition of platelet aggregability was observed at 3 hours (p=0.03), and at 6 hours (p=0.04) after r-PA treatment when compared to baseline.

After treatment with collagen, platelets aggregated significantly stronger in the r-PA group at 24 hours (p=0.003) after thrombolysis when compared with the t-PA treated group. Significant inhibition of platelet aggregability occurred at 6 hours (p=0.009) after r-PA therapy as compared to baseline.

Treatment with thrombin resulted in significantly stronger platelet aggregation significantly stronger in the r-PA group (p=0.01) at the 24 hours time point.

After treatment with Ristocetin, platelet aggregation was consistently high during the first 24 hours after thrombolysis with no statistically significant differences between and within the groups.

Flow cytometric analysis of fluorescence expression of single-labeled FITC-conjugated platelet surface receptors in AMI patients during the first twenty-four hours after thrombolysis was also analyzed. Baseline platelet receptor expression did not differ significantly between the r-PA and t-PA groups. However, thrombolysis was associated with substantial changes in receptor expression in both groups.

Platelet expression of p24 (CD 9) was not significantly different between groups at any time point. Significant (when compared to baseline) decreases of receptor expression were observed at 3 hours (p=0.004), and at 6 hours (p=0.005), followed by an increase (p=0.04) at 24 hours after r-PA therapy. Within the t-PA group, no early receptor inhibition was observed, but at 24 hours receptor expression was higher (p=0.01) than at the baseline.

Expression of glycoprotein Ib receptor on the surface of platelets was very similar between the two groups, with a delayed significant inhibition of glycoprotein Ib expression at 12 hours after both r-PA (p=0.02) and t-PA (p=0.007) therapy. However, there were no significant differences between groups.

Although the profile of glycoprotein IIb expression (slight early decrease followed by later increase) was present following both thrombolytic agents, there were no statistically significant differences between or within the groups.

A significant (when compared to baseline) increase of glycoprotein IIIa expression was observed at 12 hours (p=0.01), and at 24 hours (p=0.0003) only after r-PA, but not after t-PA therapy. However, these differences did not reach significance between the groups.

As compared to baseline, a significant early decrease of glycoprotein IIb/IIIa expression (p=0.03) was observed at 3 hours after r-PA therapy. Glycoprotein IIb/IIIa expression was elevated at 24 hours for both r-PA (p=0.002) and t-PA (p=0.035) groups. Moreover, the extent of platelet glycoprotein IIb/IIIa expression was significantly higher in the r-PA group at 24 hours (p=0.037) after thrombolysis when compared with the t-PA treated group.

The only significant difference in platelet VLA-2 expression between and within groups was an almost three fold fluorescence intensity increase in the r-PA patients after 24 hours (p=0.04) when compared with the t-PA treated group.

Very similar profiles of changes in P-selectin expression were observed between groups. An early significant decrease (p=0.001) for the r-PA group, and (p=0.009) for the t-PA group; was followed by a significant increase in P-selectin expression (p=0.009) for the r-PA group, and for the t-PA group (p=0.02).

A significant increase (p=0.002) in fluorescence intensity for PCAM-1 was found in the r-PA patients after 24 hours when compared with the t-PA treated group. A significant decrease of PECAM-1 expression was observed at 3 hours after thrombolysis in the r-PA group (p=0.01), when compared to baseline data.

Dynamic changes in platelet vitronectin receptor expression were similar between groups, and revealed a significant increase at 12 hours after thrombolysis for the r-PA group (p=0.04) and for the t-PA treated patients (p=0.04). At 24 hours after thrombolysis, vitronectin receptor expression was even higher in the r-PA group (p=0.008), whereas the t-PA group levels trended toward the baseline.

### C. Example 3

### Effect of Tenecteplase Versus Alteplase on Platelets During the First Three Hours of Treatment for Acute Myocardial Infarction: the Assessment of the Safety and Efficacy of a New Thrombolytic Agent (ASSENT-2) Platelet Substudy

The example demonstrates the direct effects of tenecteplase and alteplase on platelet function. The effects of these agents were compared by extensive functional and morphologic analysis in blood samples from human volunteers (in vitro study) and by assessment of platelet-released biomarkers in acute myocardial infarction patients in the period immediately following thrombolysis (ex vivo study).

Materials and Methods

Blood samples for the in vitro study were obtained from 9 healthy volunteers. None of the subjects smoked or had hypertension, diabetes, or an abnormal hematocrit. None had received aspirin or any other antiplatelet drugs. All subjects underwent blood sampling after at least 30 minutes of rest and 2 or more hours of fasting. Blood was drawn from an antecubital vein between 8 am and 10 am in order to avoid any diurnal influence. A 21-gauge butterfly needle was used to draw blood into a tube containing 3.8% sodium citrate (1:9 volume); the first 1.5 mL of free running blood was discarded. One tube was kept as an internal control. A second tube was incubated with tenecteplase (TNK) for 30 minutes at room temperature in order to achieve a final concentration of 12 μg/mL. The third tube was similarly incubated with alteplase (t-PA) to achieve a concentration of 4 μg/mL. The concentrations of TNK and t-PA that were chosen approximated conventional plasma levels observed in patients receiving TNK and t-PA bolus (30 mg, and 15 mg, respectively; data on file, Genentech, Inc., South San Francisco, Calif.). Fresh solutions of TNK and t-PA were prepared in Dulbecco's phosphate buffered saline (DPBS) ex tempore on the morning of the platelet studies.

For the platelet aggregation (PA) studies, platelet-rich plasma (PRP) was first isolated. Briefly, the citrate and whole blood mixture was centrifuged at 1200 g for 2.5 minutes in order to obtain platelet-rich plasma (PRP); the plasma was kept at room temperature for use within 1 hour. Platelet counts were determined for each PRP sample with a Coulter Counter ZM (Coulter Co., Hialeah, Fla.). Platelet numbers were adjusted to 3.5×10⁸/mL with homologous platelet-poor plasma. Platelet aggregation was induced by 20 μM ADP and 1 μg/mL collagen diluted in Tyrode's buffer solution (10 mM Tris, 0.15 M NaCl, pH=7.4). All agonists were obtained from the Chronolog Corporation (Havertown, Pa.). Platelet aggregation was determined by using a 4 channel Chrono-log Lumi-Aggregometer (model 560-Ca) and expressed as the maximum percentage of light transmittance change (% max) from the baseline at the end of the recording time. Platelet-poor plasma was used as a reference. Platelet aggregability curves were recorded for 6 minutes and analyzed according to internationally established standards (see, e.g., Ruggeri, Z. M. (1994) Semin. Hemat. 31:229-239).

Whole blood samples were prepared as follows: whole blood citrate mixture was diluted 1:1 with 0.5 mL Tyrode's buffer solution, then swirled gently. The cuvette with the stirring bar was placed in the incubation well and allowed to warm to 37° C. for 5 minutes. Then the sample was transferred to the assay well. An electrode was placed in the sample cuvette. Platelet aggregation was stimulated with 5 μg/mL collagen. Platelet aggregation studies were performed by using a whole blood lumi-aggregometer (model 560-Ca, Chrono-log Corporation). Platelet aggregability was expressed as the change in electrical impedance and is expressed in ohms. Aggregation curves were recorded for 6 minutes and analyzed by use of Aggrolink® software (Chrono-log Corporation).

The PFA-100™ (Dade Behring, Deerfield, Ill.) is a high shear-inducing platelet function analyzer that simulates primary hemostasis after injury to a small vessel under flow conditions (see, e.g. Kundu, S. K. et al. (1996) Clin. Appl. Thromb. Hemost. 2:241-249). The device provides a constant negative pressure that aspirates a whole blood-citrate mixture, which comes into contact with a collagen-coated membrane and then passes through an aperture. The time required to obtain occlusion of the aperture is digitally recorded and is a measure of shear-induced platelet aggregation.

With rapid platelet-function assay (RPFA (Ultegra®); Accumetrics, Inc., San Diego, Calif.), polystyrene beads coated with fibrinogen are placed in a cartridge along with a peptide that activates the thrombin receptor. A whole blood citrate mixture is added to the cartridge, and agglutination between platelets and coated beads is recorded. The data mirror turbidometric platelet aggregation and reflect the degree of platelet glycoprotein IIb/IIIa blockade.

The surface expression of platelet receptors was determined by flow cytometry by using the following monoclonal antibodies: CD 41 (glycoprotein [GP] IIb/IIIa, α IIbβ₃) CD 42b (GP Ib), CD 62p (P-selectin), CD 51/CD 61 (αᵥβ₃, or vitronectin receptor), CD 31 (platelet endothelial cell adhesion molecule [PECAM]-1), CD 107a (LAMP-1), CD 107b (LAMP-2), CD 63 (LIMP or LAMP-3), and CD 151 (PETA-3) (PharMingen, San Diego, Calif.). Platelet-leukocyte interactions were assessed by using dual antibodies for a pan-platelet marker (CD 41), together with CD 14, a monocyte/macrophage marker. The blood-citrate mixture (50 μl) was diluted with 450 μl Tris buffered saline (TBS) (10 mmol/L Tris, 0.15 mol/L sodium chloride) and mixed by gently inverting an Eppendorf tube 2 times. The corresponding antibody was then added (5 μl) and incubated at 4° C. for 30 minutes. After incubation, 400 μl of 2% buffered paraformaldehyde was added for fixation. The samples were analyzed on a Becton Dickinson FACScan flow cytometer set up to measure fluorescent light scatter, as previously described. All parameters were collected by using 4-decade logarithmic amplification. The data were collected in list mode files and then analyzed. P-selectin was expressed as percent positive cells. Other antigens were expressed as log mean fluorescence intensity.

Forty-one patients with ST-segment elevation AMI enrolled in a single ASSENT-2 site (Rashid Hospital, Dubai, United Arab Emirates) were studied. The inclusion and exclusion criteria of ASSENT-2 have been previously reported ([no authors listed] (1999) Lancet 354:716-722). Briefly, patients included in the trial were between 18 and 70 years of age and were seen within 6 hours of symptom onset with >30 minutes of continuous symptoms. Patients in whom 12-lead electrocardiography revealed >1 mm ST-segment elevation in more than 2 limb leads or >2 mm ST-segment elevation in more than 2 contiguous precordial leads, or left bundle branch block were included. Patients were excluded if they had a history of hemorrhagic diathesis, stroke, major surgery, or significant trauma within the previous 6 weeks, or hypertension of >200/110 mm Hg. After informed consent was obtained, 7 serum samples were drawn. Samples were collected at baseline and at 30-minute intervals during a 180-minute period. Within 15 minutes of sample acquisition, plasma was separated by centrifugation at 800 g for 10 minutes, aspirated, placed in vials, and snapped frozen at −20° C.

Enzyme-linked immunosorbent assay (ELISA) was used according to standard sandwich techniques for CD 31 (sPECAM-1), sVACAM-1, sP-selectin, (R&D Systems Inc. Minneapolis, Minn.), beta-thromboglobulin, Asserachrom® (Diagnostica Stago Inc., Parsippany, N.J.), platelet factor 4 Asserachrom®, thromboxane, and prostacyclin (Cayman Chemical Co., MI). Each sample was measured in duplicate, and the overall intra-assay coefficient of variation was between 3.1%±0.4% and 7.6%±1.0%, with a plasma recovery rate between 89.7% and 98.7%.

For all comparisons, statistical analyses were done by using repeated measures ANOVA. Post hoc comparison was performed by use of the Bonferroni t-test to identify specific differences in platelet aggregation and receptor expression between patients treated with TNK and t-PA, and controls. The Mann-Whitney U test was used to analyze non-parametric data. Normally distributed data were expressed as mean±SE, and skewed data as median (range). Probability values of P<0.05 were regarded as statistically significant. Linear regression analysis was applied to normally distributed data for all study participants by using the Statview 4.1 program for analysis.

Results

First, in vitro comparison of TNK Versus t-PA on platelet activation and surface receptor expression was performed. Among 15 parameters measured, 10 characteristics indicate diminished platelet function for both TNK- and t-PA-treated samples as compared with baseline DPBS values. Both TNK and t-PA-induced platelet-rich plasma, and in the whole blood, reduction of the platelet activation units assessed by the Ultegra Platelet Analyzer and prolongation of the closure time with the PFA-100 instrument. Whole blood flow cytometry revealed reduced expression of glycoprotein IIb/IIIa and a trend toward diminished expression of PECAM-1, and formation of platelet-monocyte aggregates for both agents when compared with the saline-treated samples.

When comparing 2 sets of data when samples were incubated with TNK and t-PA, TNK-treated samples exhibited a statistically significant decrease of the whole blood aggregometry, platelet activation with the Ultegra analyzer, and extension of the closure time with the ADP/collagen cartridge by use of the PFA-100 device. Significant reduction of the glycoprotein IIb/IIIa, PECAM-1, vitronectin receptor, and CD 151 expression was observed in the TNK-treated samples when compared with the t-PA samples. Formation of the platelet-monocyte aggregates was also lower in the TNK group.

In the ASSENT-2 Platelet Substudy, 21 of the 41 patients enrolled received TNK (30-50 mg), while the remaining 20 received t-PA (40-85 mg). There were no substantial differences in baseline characteristics for the AMI patients.

Therapy with TNK and t-PA was associated with changes in the plasma concentrations of platelet-derived biomarkers. Overall, infusion of both agents caused a decreased release of substances from platelets. Substantial differences between agents were also observed: the group receiving TNK exhibited a stronger prevention of platelet activation than the group receiving t-PA. Plasma levels of PECAM-1 were 2 times higher in the patients with AMI than in controls and were significantly inhibited in TNK-but not in t-PA-treated patients. Vascular cell adhesion molecule-1 (VCAM-1) release, an established marker of endothelial activation, was also inhibited significantly after 90 minutes in the TNK group, with no marked changes in the t-PA group.

An immediate increase of P-selectin plasma concentrations at 30-60 minutes, followed by a significant decrease of P-selectin at 120-150 minutes with a rebound at 180 minutes was observed in the TNK group and was also shown to increase steadily in the t-PA group. Similar patterns were shown in the release of platelet factor 4 and β-thromboglobulin concentrations with TNK. Decreased concentrations of α-granule constituents were shown with t-PA, but to a lesser extent. Treatment with TNK resulted in a significant and early decrease in plasma concentrations of thromboxane and prostacyclin. Although the pattern of prostacyclin release was very similar between the agents, inhibition of thromboxane formation was significantly stronger with TNK than with t-PA therapy.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method of treating a thrombotic or thromboembolic event in a patient in need of such treatment comprising:
(a) administering a therapeutically effective amount of a thrombolytic agent; and
(b) administering a therapeutically effective amount of a platelet inhibitor, wherein said platelet inhibitor is administered to said patient not concominantly with said thrombolytic agent, but after thrombolysis has occurred.

2. A method of claim 1, wherein said thrombotic or thromboembolic event is selected from the group consisting of unstable angina, acute myocardial infarction, ischemic stroke, acute coronary ischemic syndrome, catheter thrombosis, deep vein thrombosis, any arterial vessel thrombosis, thromboembolism, thrombotic occlusion and reocclusion, transient ischemic attack, first or subsequent thrombotic stroke, Q wave myocardial infarction and ST-segment elevated myocardial infarction.

3. The method of claim 1, wherein said thrombolytic agent is selected from the group consisting of alteplase, reteplase, tenecteplase, streptokinase, pro-urokinase, urokinase, lanoteplase, monteplase, saruplase, staphylokinase and anisoylated plasminogen-streptokinase activator kinase.

4. The method of claim 1, wherein said platelet inhibitor is administered between about 6-24 hours after thrombolysis has occurred.

5. The method of claim 1, wherein said platelet inhibitor is administered between about 12-24 hours after thrombolysis has occurred.

6. The method of claim 1, wherein said platelet inhibitor is administered between about 18-24 hours after thrombolysis has occurred.

7. The method of claim 1, wherein said platelet inhibitor is administered about 20-24 hours after thrombolysis has occurred.

8. The method claim 1, wherein said platelet inhibitor is selected from the group consisting of aciximab, eptifibatide, tirofoban, lamifiban, aspirin, ticlopidine, clopidogrel, dipyridamole, aggrenox® and selective serotonin reuptake inhibitor.

9. The method of claim 1 further comprising administering concomitantly with said thrombolytic agent and said platelet inhibitor an anticoagulant compound.

10. The method of claim 1 further comprising administering concomitantly with said thrombolytic agent an anticoagulant compound.

11. The method of claim 1 further comprising administering concomitantly with said platelet inhibitor an anticoagulant compound.

12. The method of any one of claims 9-11 in which said anticoagulant compound is selected from the group consisting of unfractionated heparin, heparin and hirudin.

13. The method of claim 1 further comprising administering said thrombolytic agent multiple times.
